(19) United States
(12) Plant Patent Application Publication
(10) Pub. No.: US 2015/0052647 P1
(43) Pub. Date: Feb. 19, 2015

(54) CHAMELAUCIUM PLANT NAMED 'MARS'

(50) ;

(71) Applicant: Nitzan Nir, Kfar Hess (IL)

(72) Inventor: Nitzan Nir, Kfar Hess (IL)

(21) Appl. No.: 13/987,629

(22) Filed: Aug. 16, 2013

(51) Int. Cl. A01H 5/00

(52) U.S. Cl. PLT/229

(57) ABSTRACT

A new and distinct Chamelaucium cultivar named ‘MARS’ is disclosed, characterized by unique flower size and color, bud size, large quantity of buds and flowers, branch structure and date of blooming. The new variety is a Chamelaucium, normally producing cut flower stems, or as a garden or container plant.

### LATIN NAME OF THE GENUS AND SPECIES

Chamelaucium uncinatum

### VARIETY DENOMINATION

‘MARS’

### BACKGROUND OF THE INVENTION

The new Chamelaucium cultivar is a product of a planned breeding program conducted by the inventor, Nitzan Nir, in Kfar Hess, Israel. The objective of the breeding program was to produce new Chamelaucium varieties for ornamental commercial applications. The new variety is a product of crossbreeding that occurred during 2010 at a commercial nursery in Kfar Hess, Israel.

The seed parent is the unpatented, proprietary seedling variety Chamelaucium ‘2W140M003’. The pollen parent is the unpatented, variety Chamelaucium ‘Early Violet’. The new variety was discovered in March of 2011 by the inventor in a group of seedlings resulting from the 2010 crossing, in a commercial nursery in Kfar Hess, Israel.

Asexual reproduction of the new cultivar has been performed by vegetative cuttings. This was first performed at a commercial nursery in Kfar Hess, Israel in May of 2011 and has shown that the unique features of this cultivar are stable and reproduced true to type in five successive generations.

### SUMMARY OF THE INVENTION

The cultivar ‘MARS’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘MARS’. These characteristics in combination distinguish ‘MARS’ as a new and distinct Chamelaucium cultivar:

1. Bright pink flower color

2. Large quantity of buds and flowers

### PARENT COMPARISON

Plants of the new cultivar ‘MARS’ are similar to plants of the seed parent, Chamelaucium ‘2W140M003’ in most horticultural characteristics, however, plants of the new cultivar ‘MARS’ produce a different flower size and bloom at a different time than the seed parent.

Plants of the new cultivar ‘MARS’ are similar to plants of the pollen parent; Chamelaucium ‘Early Violet’ in most horticultural characteristics, however, plants of the new cultivar ‘MARS’ produce a different bud size, more buds and flowers, different branch structure and different date of bloom than the pollen parent.

### COMMERCIAL COMPARISON

‘MARS’ can be compared to the Unpatented commercial variety Chamelaucium ‘M.B. Violet’, Plants of ‘MARS’ are similar to plants of ‘M.B. Violet’ in most horticultural characteristics, however, plants of ‘MARS’ produce a different bud size, more buds and flowers and different branch structure than the comparator.

‘MARS’ can be compared to the commercial variety Chamelaucium ‘Umbrella’ U.S. Plant patent application Ser. No. 13/986,493. Plants of ‘MARS’ are similar to plants of ‘Umbrella’ in most horticultural characteristics, however, plants of ‘MARS’ produce a different flower color, date of blooming and branch structure than the comparator.

### BRIEF DESCRIPTION OF THE PHOTOGRAPH

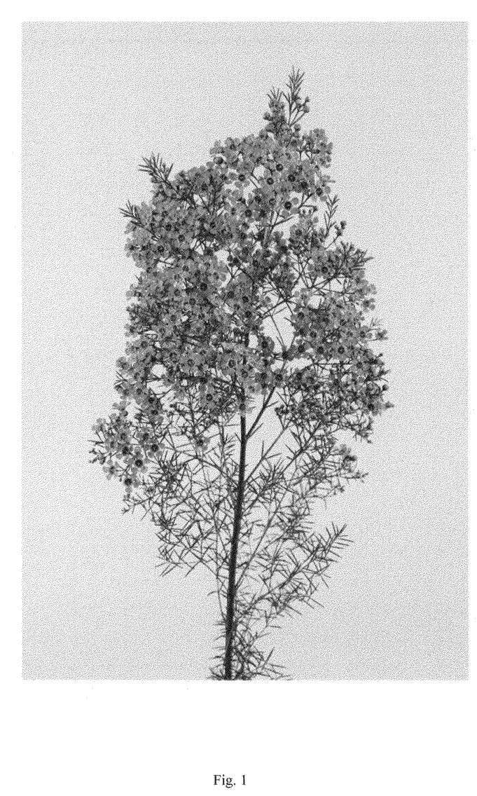

The accompanying photograph in FIG. 1 illustrates in full color a close up of typical flowers and foliage of ‘MARS’. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘MARS’ plants grown outdoors in Kfar Hess, Israel. The growing temperature ranged from 15° C. to 35° C. during the day and from −2° C. to 35° C. during the night. General light conditions are bright, normal sunlight. Measurements and numerical values represent averages of typical plant types. Chemical treatments included general fungicides and insecticides.
Botanical classification: Chamelaucium uncinatum ‘MARS’
Propagation:
   Time to produce a rooted plantlet.—30 to 60 days at approximately 24° C.
   Root description.—Fibrous.
Plant:
   Age of plant described.—2 months from a rooted cutting.
   Growth habit.—.
   Pot size of plant described.—2 liter pot.
   Height.—200 cm to top of foliage: 199 cm to top of flowers.
   Plant spread.—80 cm.
   Growth rate.—Rapid.
   Branching characteristics.—Free branching.
   Length of primary lateral branches.—60 cm and on.
   Diameter of lateral branches.—0.4 cm.
   Quantity of primary lateral branches.—120-150.
   Characteristics of primary lateral branches.—Form: Round Diameter: 0.5 cm. Color: Near RHS Yellow-Green 147A Texture: Smooth Strength: Stems brittle.
   Internode length.—1.5-5 cm.
Foliage:
   Leaf.—Arrangement: Opposite Quantity: Approximately 6 leaves, in the 10-15 cm section from the top of the branch . Average Length: 1.5 cm. Average Width: 0.1 cm. Shape of blade: Needle Apex: Acute Base: Attenuate. Margin: Entire Texture of surfaces: All surfaces glabrous. Pubescence: None. Aspect: Straight, approximately 45 degree angle from stem. Color: Young foliage: Near RHS Yellow-Green 147B Mature foliage: Near RHS Green 137A Venation: Indistinguishable from foliage. Petiole: Not present.
Flower:
   Natural flowering season.—Plants planted during April through October begin flowering December through February, without a juvenility period. Plants flower continuously under short day conditions.
   Days to flowering from rooted cutting.—Short day photoperiodic conditions. No juvenility.
   Inflorescence type.—Panicle.
   Individual flower type.—Single, 5 petals with hypanthium.
   Persistent or self-cleaning.—Self-Cleaning.
   Rate of flower opening.—70 to 90 days from bud to fully opened flower.
   Typical flowering stems harvested per plant.—120-150.
   Vase life of flowers.—Two weeks.
   Bud.—Shape: Round Length: 0.35 cm Diameter: 0.35 cm Color: Near Red-Purple 67A.
   Inflorescence size.—Diameter: Average 15 cm. Length: Average 10 cm.
   Flower size.—Diameter: 1.5 cm. Length: 0.7 cm.
   Corolla.—Petals: Length: 0.45 cm. Diameter: 0.4 cm Quantity: 5 Texture: Smooth Apex: Obtuse rounded Base: Fused into a hypanthium Color: When opening: Upper surface: Near Red-purple 67C Lower surface: Near Red-purple 67B Fully opened: Upper surface: Near Red-purple 68A Lower surface: Near Red-purple 68A Aging: Upper surface: Near Red-purple 68B Lower surface: Near Red-purple 68B.
   Hypanthium.—Diameter: 0.7 cm. Length: 0.3 cm Color: W When opening: Upper surface: Near Red-purple 71A Lower surface: Near Red-purple 70C Fully opened: Upper surface: Near Red-purple 71A Lower surface: Near Red-purple 70C Aging: Upper surface: Near Red-purple 71A Lower surface: Near Red-purple 70C Calyx/Sepals: Quantity per flower: Single conical structure. Shape: Conic Length: 0.3 cm Width: 0.7 cm Margin: Entire Texture: Smooth.
   Peduncle.—Peduncle is the lateral branch.
   Pedicel.—Length: 0.7 cm. Diameter: 0.1 cm. Color: Near Yellow-Green 147B.
   Fragrance.—
Reproductive organs:
   Stamens.—Number: 10 Filament length: 1 mm.
   Anthers.—Shape: Oval Length: 0.25-0.5 mm Color: Near Red-Purple 70C.
   Pollen.—Not observed.
   Pistil.—Number: 1 Length: 0.45 cm Style: Length: 0.44 cm Color: Near Red-Purple 68D Stigma: Shape: Round Color: Near Red-Purple 68D.
Other characteristics:
   Seeds and fruits.—Single flower produces one fruit. When the fruit is fertile, it will produce 1 seed, occasionally 2 seeds.
   Disease/pest resistance.—Neither resistance nor susceptibility to the normal diseases and pests of Chamelaucium have been observed to date.
   Temperature tolerance.—from −2° C. to 35° C.
   Drought tolerance.—Very good tolerance for drought.

What is claimed is:

1. A new and distinct cultivar of Chamelaucium plant named ‘MARS’ as herein illustrated and described.
